(19) United States
(12) Patent Application Publication
(10) Pub. No.: US 2003/0208383 A1
(43) Pub. Date: Nov. 6, 2003

(54) Nutritional information management device and method

(76) Inventors: William Hauck, Chapel Hill, NC (US); Roger Hauck, Minneapolis, MN (US)

Correspondence Address: OPPENHEIMER WOLFF & DONNELLY LLP, 45 SOUTH SEVENTH STREET, SUITE 3300, MINNEAPOLIS, MN, 55402, US

(21) Appl. No.: 10/303,942

(22) Filed: Nov. 25, 2002

Related U.S. Application Data
(60) Provisional application No. 60/377,379, filed on May 1, 2002.

(51) Int. Cl. G06F 17/60; G06F 19/00; G01N 33/48; G01N 33/50

(52) U.S. Cl. 705/3; 702/19

(57) ABSTRACT

The present invention allows a user to track nutritional intake values and assists a user in maintaining nutritional intake goals. The invention looks up the nutritional values of foods, adds foods or food combinations to a temporary memory, maintains a daily diary of nutritional intake, generates the average nutritional intake for a selected number of days, and stores nutritional goals. In addition, the present invention allows the user to create custom meal totals, representing user-included food items and their corresponding nutritional values that the user consumes in a meal. Further, the present invention compares nutritional goals with the averages of actual nutritional intake.

This application claims the benefit of U.S. Provisional Application No. 60/377,379, filed May 1, 2002.

### BACKGROUND OF THE INVENTION

This invention relates generally to a system and method for assisting a user in tracking and maintaining nutritional intake goals. Specifically, the invention relates to a system and method for searching foodstuffs and other nutritional products from a database, selecting a desired item from the database, and maintaining a record of the nutritional values of the items selected for a predetermined period of time. The invention permits selecting nutritional intake goals and comparing actual nutritional intake against those goals.

For a variety of reasons, people's diets may be limited by certain restrictions relating to the nutritional characteristics of the food they eat such as the number of calories, the grams of fat, saturated fat, carbohydrates, and protein, and the quantity per serving. For some, eating habits are tailored to achieve a desired weight. For others, food intake is regulated by a physician's directions for maintaining or improving health. Although lifestyle and a sensible combination of diet and exercise are commonly viewed as the best ways to achieve some of these goals, many people find that they require assistance in successfully implementing an effective strategy to realize their dietary objectives.

As a result, many tools have been developed to assist in these endeavors. Specifically, a variety of electronic devices exist to help people track their daily nutritional intake. Current nutritional devices typically allow users to select foodstuffs from a database and then maintain a record of the selected items and their associated nutritional values. However, the existing devices do not serve all the needs of people desiring to track their nutritional intake. For instance, these devices often require a great deal of time for the user to locate the desired food item, to observe to associated nutritional information, and log the food item in the record. Some devices require the user to view multiple menus of food categories before allowing the user to select a food item, others display long lists of foods that the user is required to peruse in order to select a food item, and still others present the user with a cumbersome and confusing menu of foods or food-types.

Accordingly, there continues to be a need for an efficient and user-friendly device and method for searching foodstuffs from a stored list, efficiently locating and selecting the desired item from the list, and maintaining a record of the nutritional values of the items selected.

### SUMMARY OF THE INVENTION

It is an object of the present invention to provide a system and method for enabling a person to exercise care in selecting a diet by recording and tracking, in an efficient and intuitive manner, the nutritional values of foodstuffs, nutrients, minerals, and vitamins he or she consumes.

A further objective of the present invention is to provide nutritional assistance by enabling a user to easily enter additional food items and nutritional information that are not already available for the user's selection.

Another object of the invention is to minimize the cost of nutritional assistance and tracking.

The present invention allows a user to look up values of foods, add foods or food combinations to temporary memory, maintain a daily diary of nutritional intake, view the average nutritional intake for a selected number of days, and store nutritional goals. Alternatively, the present invention may compare nutritional goals with the averages of actual nutritional intake.

In a preferred embodiment, the present invention is manifested in a handheld device with a user interface, a processor, a display, a database, and random access memory (RAM). When the user begins to interact with the device, the processor causes the display screen to show a main menu that includes a plurality of user selection options. During operation of the device the user may build a meal total, which represents the food items consumed by the user in a single meal, by interacting with the user interface. As food items are added to the meal total, the device maintains a tally of the nutritional values of all foods selected by the user, including the calories and the grams of fat, saturated fat, carbohydrates, and protein. These values are stored in the memory, and, at the user's selection, the device will store the meal total in a diary for a date selected by the user. The present invention also provides the user the ability to create custom meal totals, which comprise a plurality of food items as entered by the user.

Food items may be added to the meal total in different ways. For example, in one manner the food item is pre-stored in the database and added therefrom into the meal total, and in another manner the food item is entered into the database by the user and then entered in the meal total or simply entered directly to the meal total, without entering it to the database. These manners of adding food to the meal total are accomplished through the processes “add nutritional information for stored food to meal total” and “add nutritional information for new food to database or meal total,” respectively.

To add a food item to the meal total where the item is currently stored in the database, the user selects one of the plurality of user selection options from the main menu. In response to a user selection of one such option, the invention initiates the process to add nutritional information for a stored food item to the meal total comprising the steps of: prompting the user for a stored food name; accepting user input; displaying the nutritional content of the food; receiving user input for quantity of the stored food; querying the user whether to add the nutritional content to the meal total; and adding the nutritional information to the meal total in response to a user selection.

There will be times when the user wishes to enter an unusual food into his or her diet that is not included in the database. To add a food item to the meal total where the item is not currently stored in the database, the user selects a different one of the plurality of user selection options from the main menu. In response to the selection, the invention initiates the process to add nutritional information for a new food to the meal total. This option gives the user the opportunity to add his or her unique food to the database, along with the food's associated nutritional information. Alternatively, the system will enter such user-provided foods in the memory, rather than the database, and the unique food item will be stored in RAM, along with the food's associated nutritional information.

The process of adding a unique food to the database or RAM comprises the steps of: prompting the user for the nutritional content of the new food; accepting user input; querying the user whether to add nutritional content to the database as a new food item; prompting user for the name of the new food item; and adding the food item to the database. Additionally, the user has the additional option of adding a unique food to the meal total without adding it to the database, comprising the steps of: prompting the user for the nutritional content of the new food; accepting user input; querying the user to add nutritional content to meal total; and adding the nutritional information to the meal total.

Once food items are entered into the meal total and then added to the diary for a specific date, the user has the subsequent ability to review the nutritional values stored in the diary. This allows the user to view and change the nutritional intake for a given date. Additionally, the present invention generates averages of the nutritional values stored in the diary in response to a user request. This allows the user to compare the averages of historical nutritional information against user-defined goals.

In addition to the two above-described user selections that allow the user to add food items to the meal total, the main menu provides the user with other selection options. In response to yet another user selection, the invention will initiate a process “review nutritional intake history and averages,” comprising the steps of: prompting the user to select from one of three options (view the diary, calculate averages, or input goals); in response to a user selection to view the diary nutritional information, displaying the diary information for a date; and accepting user input, thereby causing the device to display diary nutritional information for the date selected by the user.

Alternatively, in response to a user selection to calculate averages in the “review nutritional intake history and averages” process, the invention performs the steps of: prompting the user for the number of days the calculated average will represent; accepting from the user the number of days the average will include; and displaying the average nutritional intake for the number of days specified by the user.

In yet another alternative, in response to the user selection to input goals, the invention performs the alternative steps of: prompting the user for the daily nutritional intake goals including calories, fat, saturated fat, carbohydrates, and protein; accepting from the user the information; and storing the information in the memory. In response to another user command, the invention displays a comparison between actual nutritional consumption and daily goals.

The invention further provides the user with the ability to adjust date and time information. To accomplish this, the user selects a yet another one of the plurality of user selection options from the main menu. In response, the invention will initiate the process to adjust the date/time information.

The foregoing and other objects, features, and advantages of the invention will become more readily apparent from the following detailed description of the preferred embodiment of the invention that proceeds with reference to the accompanying drawings.

### BRIEF DESCRIPTION OF THE DRAWINGS

FIG. 1 is a schematic drawing illustrating a preferred embodiment of the present invention;

FIG. 1A is a graphical illustration of an exemplary user interface;

FIG. 2 is a flow chart illustrating the methodology according to the present invention;

FIG. 2A is a graphical illustration of the device display showing the main menu;

FIG. 3 is a flow chart illustrating the device methodology for adding nutritional information for stored foods to the meal total;

FIG. 3A is a graphical illustration of the device display while adding nutritional information for stored foods to the meal total;

FIG. 4 is a flow chart illustrating the device methodology for adding nutritional information for new foods to the database or meal total;

FIG. 4A is a graphical illustration of the device display while adding nutritional information for new foods to the database or meal total;

FIG. 5 is a flow chart illustrating the device methodology for managing the meal total and adding it to the diary;

FIG. 5A is a graphical illustration of the device display while managing the meal total and adding it to the diary;

FIG. 6 is a flow chart illustrating the device methodology for reviewing nutritional intake history and averages;

FIG. 6A is a graphical illustration of the device display while reviewing nutritional intake history and averages;

FIG. 7 is a flow chart illustrating the device methodology for adjusting date and time information;

FIG. 7A is a graphical illustration of the device display while for adjusting date and time information.

### DETAILED DESCRIPTION OF PREFERRED EMBODIMENTS

Various embodiments of the present invention are shown and described to search and maintain nutritional values of foodstuff. It is to be understood that although these embodiments are shown and described in the context of a handheld device, various features of each embodiment can be combined with the others to produce a variety of embodiments or manifest in the form of another device.

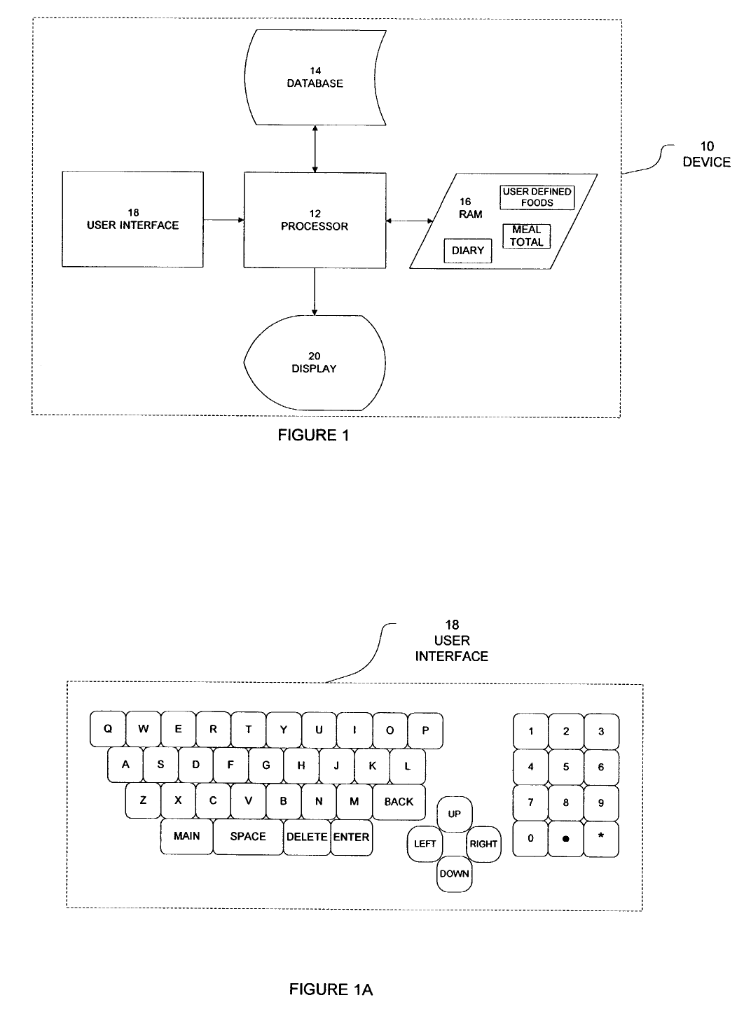

Referring now to FIG. 1, there is shown a schematic drawing of a device 10 which appropriately performs the necessary functions. Device 10 includes a processor 12, a database 14, a RAM 16, a user interface 18, and a display 20. One core component of device 10 is processor 12, which communicates with and controls other components of device 10, also shown in FIG. 1. Database 14 stores food items, their names, and their associated nutritional values. The nutritional values include the calories, the total grams of fat, saturated fat, carbohydrates, and protein per serving for each food item. The food list stored in database 14 includes permanently stored food items and, in one embodiment, new user-defined food items. RAM 16 temporarily stores information. Among other things, RAM 16 stores nutritional values constituting the user's “meal total” for a specific day and a diary. RAM 16 also stores the diary, which includes historical nutritional values for up to 31 days, and may also include future nutritional values for up to 7 days. Further, in an alternative embodiment, user-defined foods may be stored in RAM 16 rather than, or in addition to database 14.

Device 10 delivers information to the user via the display 20, which may include a monitor, by showing information to and requesting information from the user. Device 10 further includes a user interface 18 for allowing the user to enter data to device 10 for receiving and processing. FIG. 1A is a graphical illustration of one embodiment of user interface 18.

The embodiment of user interface 18 shown in FIG. 1A includes a plurality of buttons which allow the user to enter information to device 10. The buttons displaying letters, when pressed, allow the user to enter food names to device 10. The number buttons allow the user to enter numerical values to device 10, such as a number of calories, or a number of grams of fat, saturated fat, carbohydrates, or protein per serving. The ‘main,’ ‘back,’ and direction buttons allow the user to navigate device 10 and to accomplish different functions.

The particular embodiment of user interface 18 is not limited to a keyboard as shown in FIG. 1A. It is contemplated that other embodiments of user interface 18 may be used. For example, user interface 18 may include a touchscreen, a mouse, a joystick, or speech recognition capabilities.

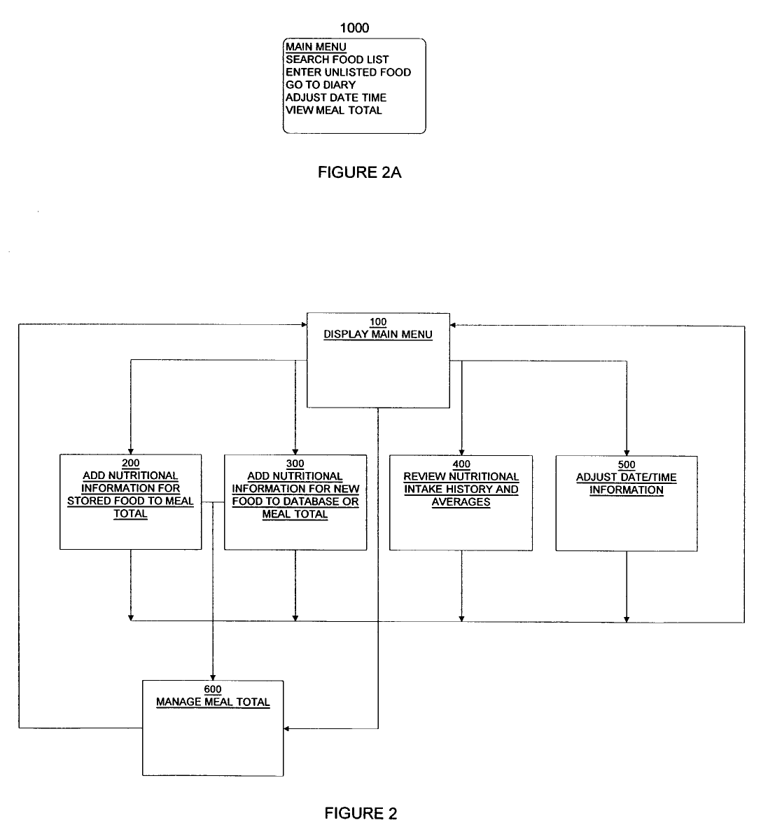

Referring now to FIG. 2, there is a flow chart illustrating a session with the user interacting with device 10. When the user begins to interact with device 10—either by pressing a button on user interface 18 or turning on device 10—device 10 displays to the user a main menu 1000, shown in FIG. 2A, with a plurality of options for the user's selection, step 100. Main menu 1000 may include many options. For example, these options may include: SEARCH FOOD LIST, ENTER UNLISTED FOOD, GO TO DIARY, ADJUST DATE TIME, and VIEW MEAL TOTAL as shown in the example display of main menu 1000. Each of the options represent high-level steps performed by device 10, shown in FIG. 2 as steps 200, 300, 400, 500, and 600.

All of the above-indicated high-level steps comprise a plurality of sub-steps, as described below. Both steps 200 and 300, among other things, allow the user to build a meal total, which is a tally of the nutritional values of foods selected by the user for a specific meal or day. Step 200, “add nutritional information for stored food to meal total,” allows the user to add to the meal total a food item currently stored in database 14. On the other hand, step 300, “add nutritional information for new food to database or meal total,” allows the user to add to the meal total a new, user-defined, food item. Step 300 also allows the user to accomplish a different operation: adding a new, user-defined item to database 14 or RAM 16 for future use.

Once the user has implemented steps 200 and 300 to add food items to the meal total, step 600, “manage meal total,” allows the user to manage the meal total, including naming a meal total, making changes to the meal total, and adding the meal total to the diary. The user may initiate step 600 from within step 200 or 300, as shown in FIG. 2.

Step 400, “review nutritional intake history and averages,” generally allows the user to review information that has been added to the diary, generate average nutritional intakes, set nutritional intake goals, and compare average nutritional intakes and nutritional intake goals. Step 500, “adjust date/time information,” allows the user to make corrections to the date and time information.

The user has the ability to navigate the options displayed on the main menu by interacting with user interface 18 to select a high-level step from the main menu 1000. Standard navigation of device 10 involves presenting a plurality of options to the user or device 10 requesting information from the user and then accepting input from the user. When device 10 displays options to the user on display 20, one option is highlighted. In order to choose between options and select the desired one, device 10 changes which option is highlighted in response to the user pressing the UP and DOWN buttons. The option above or below the currently highlighted item is highlighted in response to the user pressing the UP or DOWN button on user interface 18 respectively. Once the desired option is selected, the user may communicate that fact to device 10 by pressing the ENTER button. In response to the ENTER button being pressed, device 10 accepts and processes the user's selection. The user can also cause device 10 to return to the previous display screen by pressing the BACK button. Further, device 10 returns to step 100 and displays the MAIN MENU at any time in response to the user pressing the MAIN button. On appropriate screens, the user can enter text and numbers by using the proper letter or number buttons on user interface 18. The user can correct mistakes by pressing LEFT button, which deletes previous entries; each press of LEFT deletes the previous letter until no letters remain.

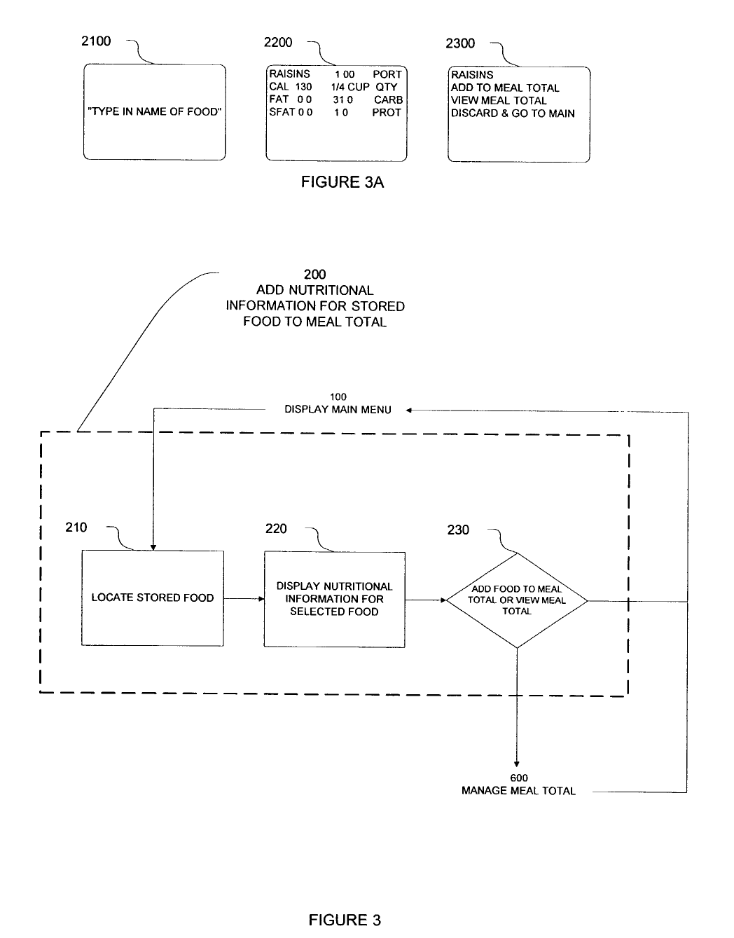

Returning to the main menu display, step 100, in response to the user's selection of the SEARCH FOOD LIST option device 10 begins ADD NUTRITIONAL INFORMATION FOR STORED FOOD TO MEAL TOTAL, step 200. The first step is locating a stored food, step 210, as shown on FIG. 3. Device 10 prompts the user to enter the name of a food item. Example display 2100 shown in FIG. 3A demonstrates the manner in which device 10 may request the information from the user.

In response to a request from device 10, the user may enter a food item name with user interface 18. As the user begins typing the food name, device 10 causes display 20 to automatically show foods starting with the letters typed thus far, updating the display after each keystroke. For example, if the user intends to enter the food “raisins,” after typing “r” the display might show “radishes,” but after typing “rai” display 20 shows “raisins.” To maintain a user-friendly display, this search procedure is displayed on a single screen-not through multiple menus-and only a single food item is displayed at a time, thereby maintaining simplicity for the user.

Once the user has located the desired food item, pressing ENTER causes device 10 to accept that particular food item and proceed to the next step, “display nutritional information for selected food,” step 220. During this step, device 10 displays the nutritional values associated with the selected food item in the previous step. For example, display 2200 demonstrates the display screen where the selected food is raisins. After the user presses ENTER, device 10 proceeds to the next step.

In an additional aspect of the present invention, device 10 further accepts from the user input of the number of portions the user would like to select. The default is one portion. If the user chooses to enter a different number of portions, the displayed nutritional values update automatically to accurately reflect the number or portions the user has selected.

During the subsequent step, “add food to meal total or view meal total,” step 230, the user may accomplish three general operations: (1) add the selected food item to the meal total; (2) view the meal total without adding the selected food item to the meal total; or (3) return to the main menu without adding the selected food item to the meal total. In step 230, the device 10 displays the selected food item and prompts the user to select from a plurality of options. These options may include: “ADD TO MEAL TOTAL,” “VIEW MEAL TOTAL,” and “DISCARD & GO TO MAIN.” Example display 2300 demonstrates the display screen where device 10 prompts the user to select an option. As described above, the MEAL TOTAL is a tally of the nutritional values of foods selected by the user for a specific meal or day. The MEAL TOTAL is stored in RAM 16 and the user has the ability to add multiple foods to the MEAL TOTAL. Once the user has completed a MEAL TOTAL, that information may be added to the DIARY, which includes historical nutritional values for a user-defined period, for example, up to 31 days. The DIARY may also include future nutritional values for a user-defined period, for example, up to 7 days. Like the MEAL TOTAL, the DIARY is also stored in RAM 16, but the DIARY stores nutritional values for a plurality of days, whereas the nutritional values stored in the MEAL TOTAL are added to a particular day in the DIARY.

To accomplish the first operation, adding the selected food item to the meal total, the user selects “ADD TO MEAL TOTAL”. In response to the user's selection of ADD TO MEAL TOTAL, device 10 adds the nutritional information of the selected food item to the MEAL TOTAL and proceeds to step 600, “manage meal total,” described below.

To accomplish the second operation, viewing the meal total without adding the selected food item to the meal total, the user selects “VIEW MEAL TOTAL”. In response to the user's selection of VIEW MEAL TOTAL, device 10 proceeds to step, “manage meal total,” step 600 described below, without adding the nutritional information of the selected food item to the MEAL TOTAL.

To accomplish the third operation, returning to the main menu without adding the selected food item to the meal total, the user selects “DISCARD & GO TO MAIN”. In response to the user's selection of DISCARD & GO TO MAIN, device 10 returns to the beginning of step 100 and displays the main menu, without adding the nutritional information of the selected food item to the MEAL TOTAL. This option is particularly useful for a user that desires to merely lookup a food's nutritional information without adding it to the MEAL TOTAL.

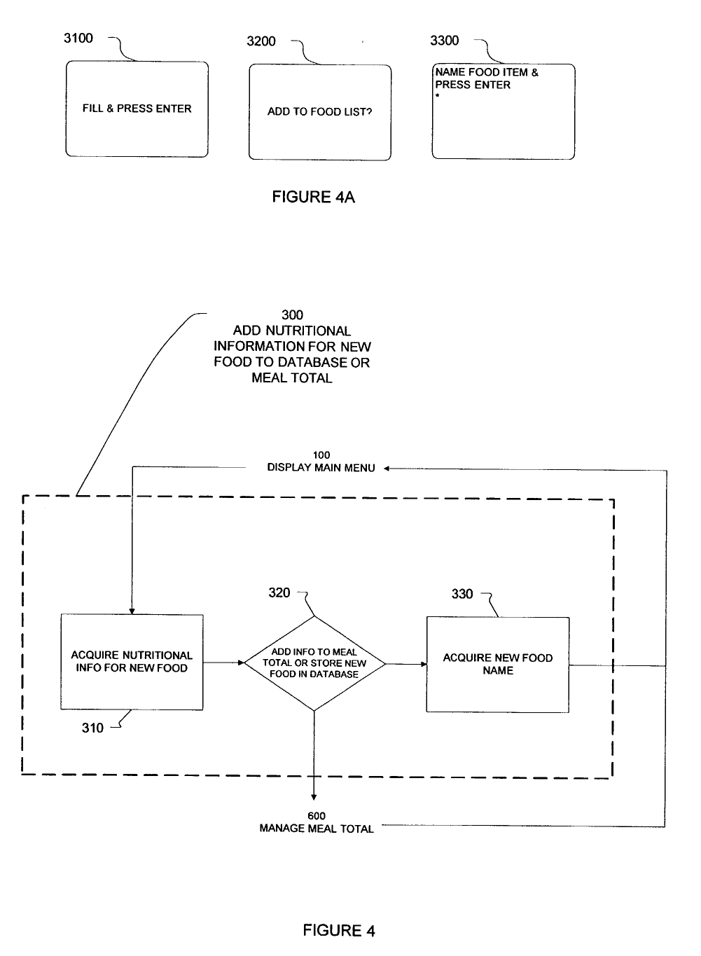

Returning to FIG. 2 and the main menu display, step 100, in response to the user's selection of the ENTER UNLISTED FOOD option, device 10 begins ADD NUTRITIONAL INFORMATION FOR NEW FOOD TO DATABASE OR MEAL TOTAL, step 300. This is an alternative procedure to add a food item to the meal total or to the database for future use, i.e. adding a new, user-defined food. Further, it is to be understood that in one embodiment such a user-defined food and its associated nutritional information is stored in the food list on database 14, or, in an alternative embodiment, such a user-defined food and its associated nutritional information is stored in RAM 16, specifically “user-defined foods.” The former embodiment, storing user-defined foods in database 14, will be described below, but this description is intended to encompass the alternative embodiment of storing user-defined foods in RAM 16 as well. As shown in FIG. 4, the first step is acquiring nutritional information for the new food, step 310. Device 10 prompts the user to enter the nutritional information of the new food item. Example display 3100, shown in FIG. 4A, demonstrates the manner in which device 10 may request the information from the user, “FILL & PRESS ENTER.”

The user may enter the nutritional values for the new food item, for example, calories, fat, saturated fat, carbohydrates, and protein. The user can navigate from field to field for each of these pieces of information by using the UP and DOWN buttons. When the nutritional information has been entered to the user's satisfaction, the user may press the ENTER button to proceed to the next screen. In response to pressing ENTER, device 10 prompts the user to determine whether to add the nutritional information to the food list, step 320. During this step, the user may accomplish two general operations: (1) add the nutritional information to the meal total without creating a new food that is stored in the database, or (2) create a new food item and add it to the database, but not add it to the meal total. As shown in FIG. 4A, display 3200 demonstrates the manner in which device 10 may prompt the user for a selection, “ADD TO FOOD LIST?”

To accomplish the first operation, adding the nutritional information to the meal total without creating a new food that is stored in the database, the user selects “NO”. Upon the user selecting NO, device 10 adds the nutritional information to the MEAL TOTAL without saving the nutritional information values as a custom food item in the FOOD LIST and proceeds to step 600, “manage meal total.”

To accomplish the second operation, creating a new food item and adding it to the database but not to the meal total, the user selects “YES”. Upon the user selecting YES, device 10 saves the entered nutritional information in database 14 and acquires a name for the new food, step 330. This option allows the user to create a custom food entry and associate nutritional information with that food.

To accomplish custom food creation, device 10 prompts the user with NAME FOOD ITEM & PRESS ENTER, as shown in example display 3300, FIG. 4A. Custom food list items may begin with an indicator, such as an asterisk (*), to distinguish them from other foods that are permanently stored in database 14. Custom food items may be deleted while they are being displayed by highlighting the food item and pressing DELETE. After the user enters a name for the custom food item and presses ENTER, device 10 stores it in database 14. Upon storing the custom food item, device 10 displays to the user “SAVED,” thereby indicating that it has been successfully saved, and return to step 100.

Returning to FIG. 2 and the main menu display, step 100, in response to the user's selection of the VIEW MEAL TOTAL option, device 10 begins MANAGE MEAL TOTAL, step 600. While the user may select other options to initiate step 600 as described above, selecting VIEW MEAL TOTAL allows the user to immediately see the contents of the MEAL TOTAL. This option is particularly useful when the user wants to view the MEAL TOTAL without initiating steps 200 or 300.

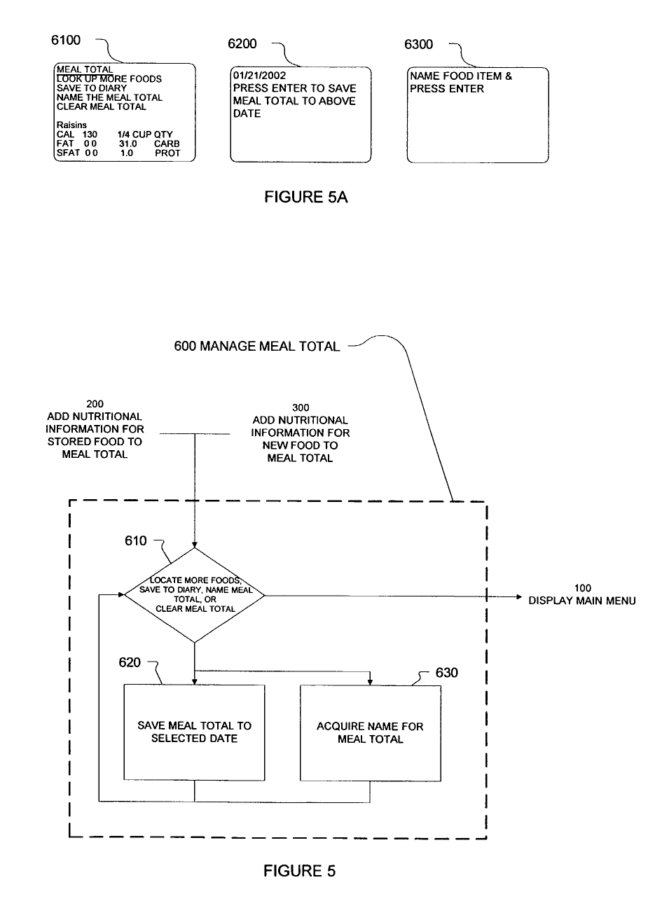

As shown in FIG. 5, the first step of process 600 displays the current MEAL TOTAL and provides the user a plurality of options, step 610. Example display 6100, shown in FIG. 5A, demonstrates the manner in which device 10 may display the MEAL TOTAL and request the information from the user. If the user has just added nutritional information to the MEAL TOTAL, the current display reflects that addition.

On the currently displayed screen, the user has the opportunity to navigate through the food items that comprise the nutritional content of the meal total by pressing the DOWN arrow. The foods are displayed on the screen, preferably one at a time. While navigating through these food items, the user may remove any of them by pressing DELETE when the desired food is highlighted. In response to the user pressing DELETE, device 10 causes the nutritional values in the MEAL TOTAL to adjust automatically to reflect the change in the food items.

During step 610, shown in FIG. 5, the user is offered options: “LOOK UP MORE FOODS,” “SAVE TO DIARY,” “NAME THE MEAL TOTAL,” “CLEAR MEAL TOTAL.” In response to the user's selection of LOOK UP MORE FOODS, device 10 proceeds to step 100 and displays the main menu. In response to the user's selection of SAVE TO DIARY, device 10 proceeds to step 620, “save meal total to selected date.” In this step, device 10 displays the current date and prompts user with message, “PRESS ENTER TO SAVE MEAL TOTAL TO ABOVE DATE,” as shown in example display 6200. The user has option to save the meal total to the current date or changing the date using the LEFT and RIGHT buttons to highlight the month, day, or year and the number buttons to enter the appropriate number in a specified format, such as mm/dd/yyyy. In response to the user's pressing ENTER, device 10 saves the meal to diary in RAM 16 for the selected date. Device 10 displays to the user “SAVED,” thereby indicating that the MEAL TOTAL has been successfully saved to the diary, and return to step 610.

In response to the user's selection of NAME THE MEAL TOTAL, device 10 proceeds to step 630, where the user is given the opportunity to save the nutritional information currently stored as the MEAL TOTAL as a custom food item. Device 10 prompts the user with, “NAME FOOD ITEM & PRESS ENTER,” as shown in example display 6300. The user may enter a name for the custom food item and presses ENTER. In response to the user pressing ENTER, device 10 stores the custom food entry in RAM 16. Upon saving the custom food item, device 10 displays to the user “SAVED,” thereby indicating that it has been successfully saved, and return to step 610.

In response to the user's selection of CLEAR MEAL TOTAL, device 10 clears the MEAL TOTAL, erasing all of the values stored as the MEAL TOTAL from RAM 16. Device 10 then proceeds to step 100 and displays the main menu.

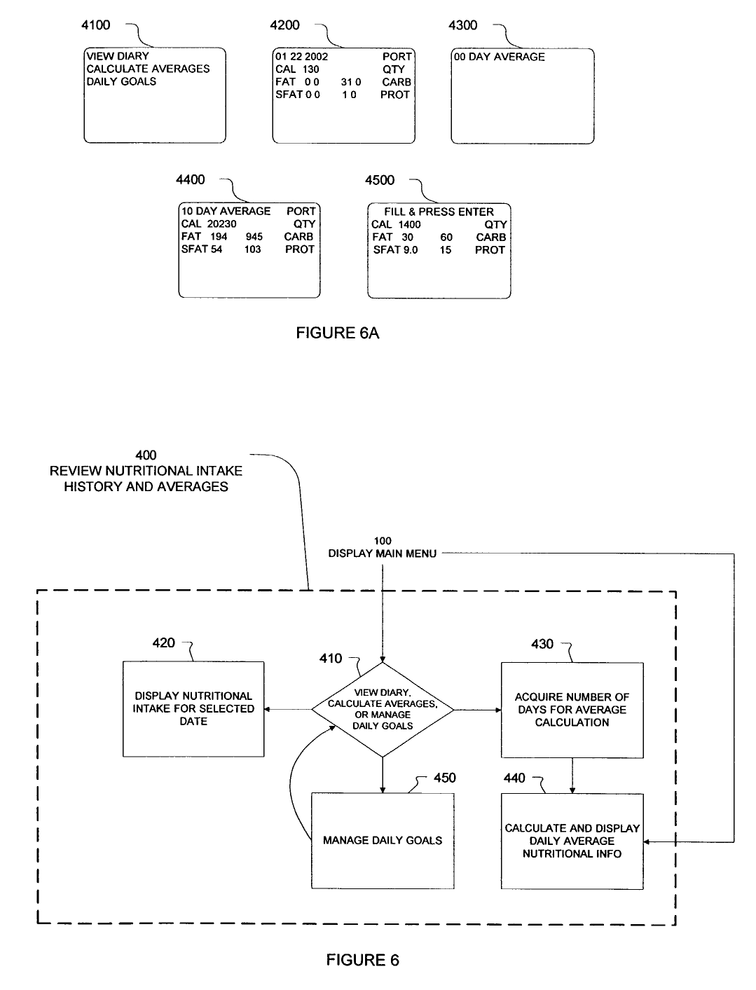

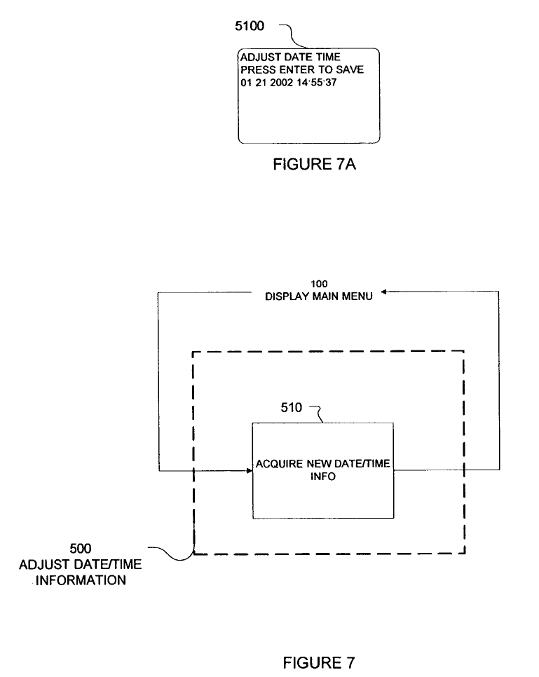

Returning to displaying the main menu, step 100, as shown in FIG. 2, in response to the user's selection of the GO TO DIARY option, device 10 begins REVIEW NUTRITIONAL INTAKE HISTORY AND AVERAGES, step 400. The first step prompts the user to select from a plurality of options, step 410 on FIG. 6. The user is prompted with such options as, “VIEW DIARY,” “CALCULATE AVERAGES,” and “DAILY GOALS,” as shown in example display 4100. In response to the user's selection of VIEW DIARY, device 10 retrieves nutritional intake information from the diary in RAM 16 and displays that nutritional intake information for the selected date, step 420. The display includes all nutritional information stored for that date as shown in example display 4200. The default date is the current date, though the user may scroll through dates using the RIGHT and LEFT buttons. The user may also adjust any of the displayed nutritional values by scrolling to the desired value using the UP/DOWN buttons and entering a new value with the number buttons. The user may save these changes by pressing the ENTER button. In response to the user pressing ENTER, the device displays to the user SAVED, thereby indicating that it has been successfully saved to RAM 16.

From step 410, in response to the user's selection of CALCULATE AVERAGES, device 10 obtains the number of days for the average calculation, step 430. To accomplish this, device 10 prompts the user with, “00 DAY AVERAGE,” as shown in example display 4300. In response, the user may indicate the number of days the average should include by entering a number between 2 and 31. In response to the user's entering a number and pressing ENTER, device 10 calculates and displays the daily average nutritional information, step 440. As shown in example display 4400, device 10 indicates the average calories, fat, saturated fat, carbohydrates, and protein the user entered into device 10 for the selected number of days. Additionally, the user may compare a daily average nutritional consumption with DAILY GOALS by pressing the * button while viewing averages.

An additional aspect of the present invention relates to another method for comparing nutritional consumption with DAILY GOALS. From step 100, the main menu display, in response to the user's pressing the * button, device 10 displays a comparison of the DAILY GOALS and the nutritional information of the food items consumed thus far that day. This aspect of the present invention, shown in FIG. 6, allows the user to easily view his or her daily nutritional status relative to his or her daily goal, directly from the main menu display, step 100, by simply pressing a single button.

From step 410, in response to the user's selection of DAILY GOALS, device 10 enables the user to manage daily goals, step 450. This option allows the user set nutritional goals—maximum daily intake of calories, fat, saturated fat, carbohydrates, and protein—as shown in example display 4500. Device 10 prompts the user with, “FILL & PRESS ENTER.” The user has the opportunity to enter a number for each of the nutritional values by navigating through the display and entering values with the number buttons. The user may save these goals by pressing the ENTER button on user interface 18. In response to the user's pressing ENTER, device 10 saves these goals to RAM 16 and display to the user “SAVED,” thereby indicating that it has been successfully saved, and return to step 410.

Returning to FIG. 2 and the main menu display, step 100, in response to the user's selection of the ADJUST DATE TIME option device 10 begins ADJUST DATE/TIME INORMATION, step 500. This step comprises acquiring new date/time information, step 510. Device 10 prompts the user with, “ADJUST DATE TIME; PRESS ENTER TO SAVE,” and display the current date/time, e.g.: “01 21 2002 14:55:37,” as shown in example display 5100. The user may adjust the date and time by navigating to the values that need correction with the LEFT and RIGHT buttons and entering new values using the number buttons. Once the values display the correct date and time, the user may save these adjustments by pressing the ENTER button. In response to the user's pressing ENTER, device 10 displays to the user SAVED, thereby indicating that the information has been successfully saved to RAM 16, and return to the main menu, step 100.

Those skilled in the art will further appreciate that the present invention may be embodied in other specific forms without departing from the spirit or central attributes thereof. In that the foregoing description of the present invention discloses only exemplary embodiments thereof, it is to be understood that other variations are contemplated as being within the scope of the present invention. For example, the present invention may be implemented on an individual computer, computers connected through a network such as the Internet, a handheld computing device such as a PDA, or a mobile telephone. Accordingly, the present invention is not limited in the particular embodiments which have been described in detail therein. Rather, reference should be made to the appended claims as indicative of the scope and content of the present invention.

What is claimed is:

1. A method in a computer system for tracking and analyzing food consumption of a user, the method comprising:
searching a system database containing a plurality of stored food items based on a user-provided search condition, the user-provided search condition including a plurality of characters, each of the plurality of characters being entered into the computer system individually such that a singular search result is displayed that satisfies each of the plurality of characters that have been entered, the singular search result being selectable by the user, wherein the singular search result is updated in response to the entry of each of the plurality of characters;
selecting a plurality of food items from the plurality of stored food items, the plurality of selected food items representing dietary consumption of the user; and
maintaining in a system memory a plurality of nutritional values and a food item consumption time, the plurality of nutritional values corresponding to the plurality of selected food items.

2. The method of claim 1, wherein the method further comprises generating an average nutritional consumption for a predetermined period, wherein the average nutritional consumption is based on the plurality of nutritional values and the food item consumption times in the system memory.

3. The method of claim 2, wherein the method further comprises:
identifying a target nutritional consumption for the predetermined period; and
comparing the average nutritional consumption against the target nutritional consumption.

4. The method of claim 1, wherein the plurality of stored food items contained in the system database include both pre-defined food items and user-defined food items.

5. The method of claim 1, wherein the method further comprises adding a user-defined food item to the plurality of stored food items contained in the system database.

6. The method of claim 5, wherein the method further comprises:
entering a plurality of user-defined values, the plurality of user-defined values representing nutritional information of the user-defined food item; and
associating the plurality of user-defined values with the user-defined food item.

7. The method of claim 1, wherein selecting the plurality of selected food items further includes identifying a serving quantity for each of the plurality of selected food items.

8. The method of claim 1, wherein the computer system includes a touchscreen for entering the plurality of characters.

9. The method of claim 1, wherein the computer system includes a voice recognition functionality for entering the plurality of characters.

10. A computer system for tracking and analyzing nutritional consumption of a user, comprising:
a database for maintaining a plurality of stored food items, each of the plurality of stored food items having nutritional information associated therewith;
a user interface for receiving a search request from the user, the search request including a plurality of characters, such that the user interface receives each of the plurality of characters individually;
a processor for accepting the plurality of characters from the user interface and querying the database therewith, the processor causing to be communicated to the user a singular search result that satisfies each of the plurality of characters that have been received by the user interface, wherein the search result is updated in response to the entry of each of the plurality of characters; and
a system memory for storing the search result upon selection by the user, the search result representing a dietary consumption of the user, the system memory for additionally storing a corresponding consumption time.

11. The computer system of claim 10, wherein the nutritional information includes at least one of the group comprising: number of calories, grams of fat, grams of saturated fat, grams of carbohydrates, and grams of protein.

12. The computer system of claim 10, wherein processor space is allocated to the system memory and causes to be stored therein a user-defined food set, the user-defined food set comprising a plurality of selected food items that are selected by the user, the user-defined food set further being selectable by the user in response to which the plurality of selected food items are stored in the system memory.

13. The computer system of claim 10, wherein the processor is configured to execute logic that generates an average nutritional consumption for a predetermined period, such that the average nutritional consumption is based on the dietary consumption of the user.

14. The computer system of claim 13, wherein:
the user interface further includes an input device for receiving a target nutritional consumption for the predetermined period; and
the processor further receives the target nutritional consumption from the user interface and executes logic to compare the average nutritional consumption with the target nutritional consumption.

15. The computer system of claim 10, wherein the database is configured and storage space therein is allocated for maintaining both pre-defined food items and user-defined food items.

16. The computer system of claim 10, wherein:
the user interface further includes an input device for receiving a user-defined food item; and
the processor further receives the user-defined food item from the user interface and allocates space in the database and stores therein the user-defined food item.

17. The computer system of claim 16, wherein:
the user interface is configured to receive a plurality of user-defined values, the plurality of user-defined values representing nutritional information of the user-defined food item; and
the processor is configured to receive the plurality of user-defined values from the user interface and executes logic to associate the plurality of user-defined values with the user-defined food item.

18. The computer system of claim 10, wherein the processor further allocates space in the system memory and causes to be stored therein a serving quantity for the search result selected by the user.

19. The computer system of claim 10, the computer system further comprising a display unit for displaying information to the user, the display unit being controlled by the processor.

20. The computer system of claim 10, wherein the user interface includes a touchscreen for receiving the plurality of characters and for displaying information to the user.

21. The computer system of 10, wherein the user interface includes a voice recognition apparatus for receiving the plurality of characters.
